(19) United States
(12) Patent Application Publication
(10) Pub. No.: US 2023/0162719 A1
(43) Pub. Date: May 25, 2023

(54) System And Method For Measuring Human Intention

(71) Applicant: Cerebian Inc., Toronto (CA)

(72) Inventor: Karim AYYAD, Toronto (CA)

(21) Appl. No.: 18/151,832

(22) Filed: Jan. 9, 2023

Related U.S. Application Data
(63) Continuation of application No. PCT/CA2021/050930, filed on Jul. 7, 2021.
(60) Provisional application No. 63/049,534, filed on Jul. 8, 2020.

(51) Int. Cl. G10L 13/027; G06F 40/56; G06F 3/01

(52) U.S. Cl. CPC G10L 13/027; G06F 40/56; G06F 3/015

(57) ABSTRACT

The following describes a device for generating speech from human intent. The device comprises at least one sensor for measuring brain signals, a processor, and a wearable portion. The following also describes a method of generating speech from human intent comprising two phases. The first phase is the training phase, which encompasses methods by which data is collected, source localization, and training of the deep learning modules. The second phase comprises sensing brain and/or muscle signals, processing the brain and/or muscle signals at the deep learning modules and converting said signals into an output. The output may be text or automatically generated speech.

### CROSS-REFERENCE TO RELATED APPLICATIONS

The present application is a continuation of International PCT Application No. PCT/CA2021/050930 filed on Jul. 7, 2021, which claims priority from U.S. Provisional Application No. 63/049,534 filed on Jul. 8, 2020, both incorporated herein by reference in their entireties.

### TECHNICAL FIELD

The following relates to systems and methods for generating speech from brain and/or muscle signals

### BACKGROUND

Speech is a process by which thoughts are translated into audible sounds or words. The Broca's and Wernicke's areas are two parts of the cerebral cortex that are linked to speech. After a thought is composed, these areas engage motor neurons that send signals to muscle fibers of the mouth, face, tongue and throat to move, which as a result generates sound that is audible and articulate.

Damage to these areas of the brain may result in aphasia. Aphasia is an impairment of language, affecting the production or comprehension of speech and the ability to read or write. Aphasia is most commonly a consequence of injury to the brain; such as stroke, dementia, and paralysis.

There are two broad categories of aphasia: fluent and nonfluent, and there are several types within these groups. Damage to the temporal lobe of the brain may result in Wernicke's aphasia, the most common type of fluent aphasia. People with Wernicke's aphasia may speak in long, complete sentences that have no meaning, adding unnecessary words and even creating made-up words. Damage to the frontal lobe of the brain may result in Broca's aphasia, the most common type of nonfluent aphasia. People with Broca's aphasia may be able to comprehend what is being said but are unable to speak fluently because their brain has trouble sending signals to muscle fibers of the mouth needed to form words. This may create frustration, since they know what they want to say, but cannot compose the words the way they wish to.

Speech generating devices or Augmentative and Alternative Communication devices (AACs) that rectify speech in humans are available. They typically rely on symbol boards, choice cards, communication books, keyboards and alphabet charts.

Similarly, Brain Computer Interfaces (BCI) or Brain Machine Interfaces (BMI) are interfaces that connect the brain's neurons to machines. Traditionally, BMI/BCI researchers have tried to measure the brain's thoughts directly from areas that compose speech.

There exists a need for a speech generating device which is able to measure the muscle signals received from areas that compose speech and generate sound that is audible and articulate.

### SUMMARY

The following describes a device for generating speech from human intent. The device comprises at least one sensor for measuring brain and/or muscle signals, a processor, and a wearable portion.

The following also describes a method of generating speech from human intent comprising two phases. The first phase is the training phase, which encompasses methods by which data is collected, source localization, and training of the deep learning modules. The second phase comprises sensing brain and/or muscle signals, processing the brain and/or muscle signals at the deep learning modules and converting said signals into an output. The output may be text, or automatically generated speech, or a different media format such as image or video generated as a result of the module's understanding of the user's intent.

The following also provides a system that can measure the intention to speak and/or to generate sound.

### BRIEF DESCRIPTION OF THE DRAWINGS

Embodiments will now be described with reference to the appended drawings wherein:

FIG. 1 is an overview of the API (application programming interface);

FIG. 2 is an overview of the system for measuring human intention;

FIG. 3 is a schematic diagram of the Training Phase;

FIG. 4 is a visualization of muscle signals for spoken words;

FIG. 5 is co-registration of fMRI with EEG prior to source localization;

FIG. 6 is a diagram of the brain where the source is localized to the visual areas of the brain showing an example of source localization;

FIG. 7 is Weight Replacement Calibration;

FIG. 8 is Weight Prediction Calibration;

FIG. 9 is an image of the Neural Networks;

FIG. 10A shows a perspective view of the device for generating speech worn by a user;

FIG. 10B shows a front view of the device for generating speech worn by a user;

FIG. 10C shows a side view of the device for generating speech worn by a user;

FIG. 11A shows a left side view of the device for generating speech in isolation;

FIG. 11B shows a ride side view of the device for generating speech in isolation; and

FIG. 11C shows a perspective view of the device for generating speech worn in isolation.

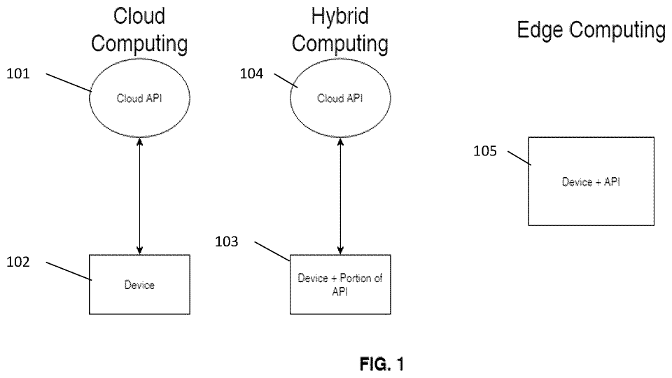

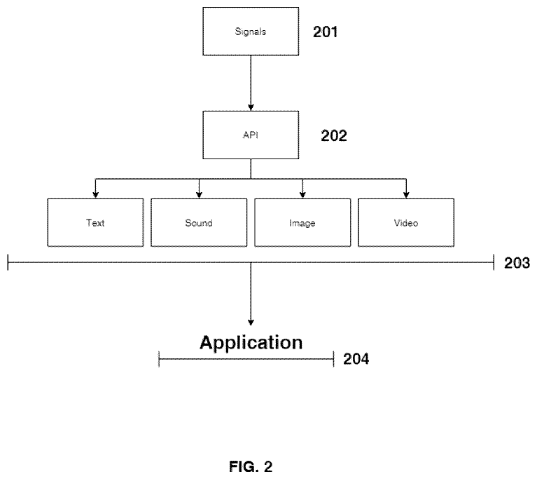

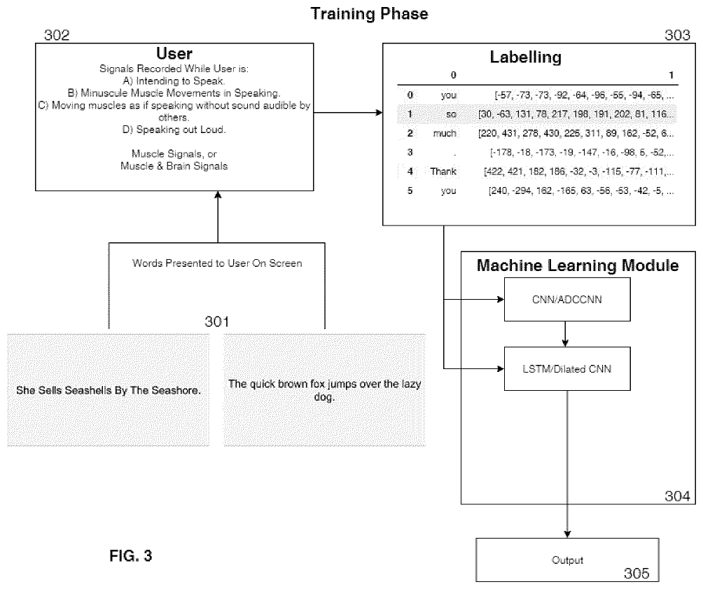

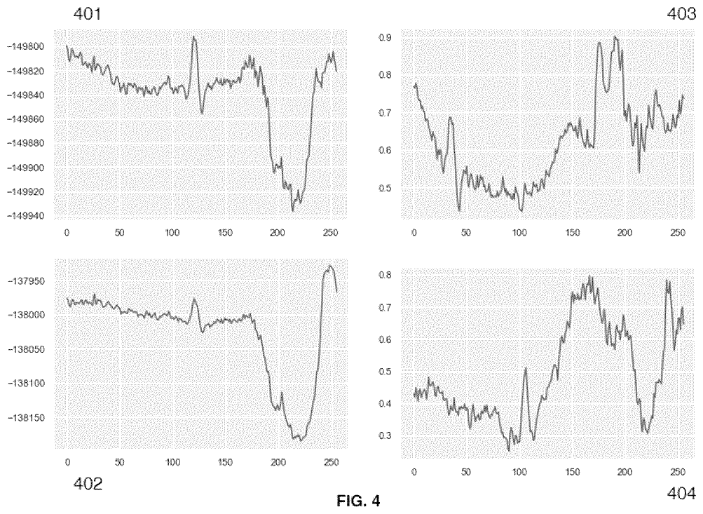

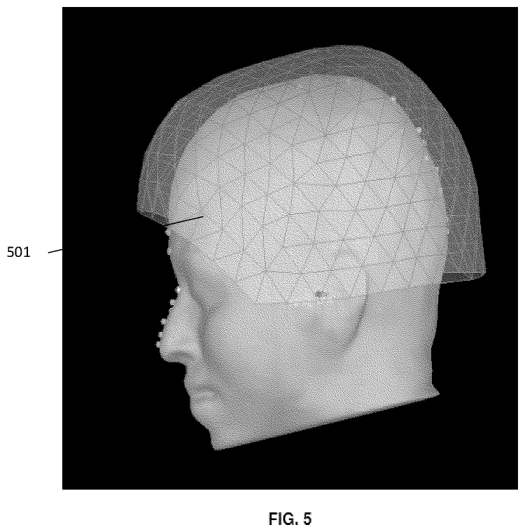

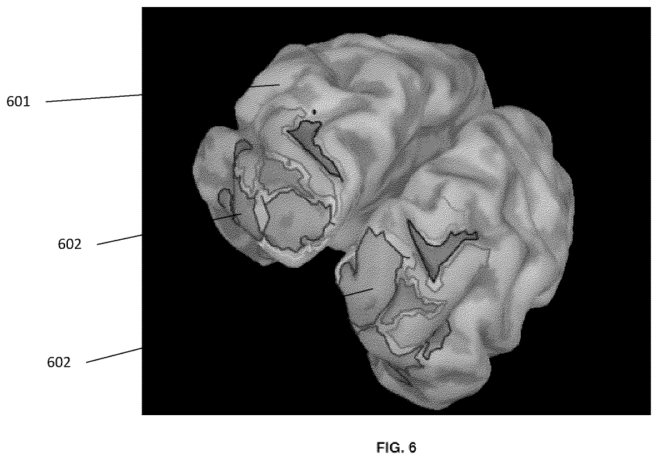

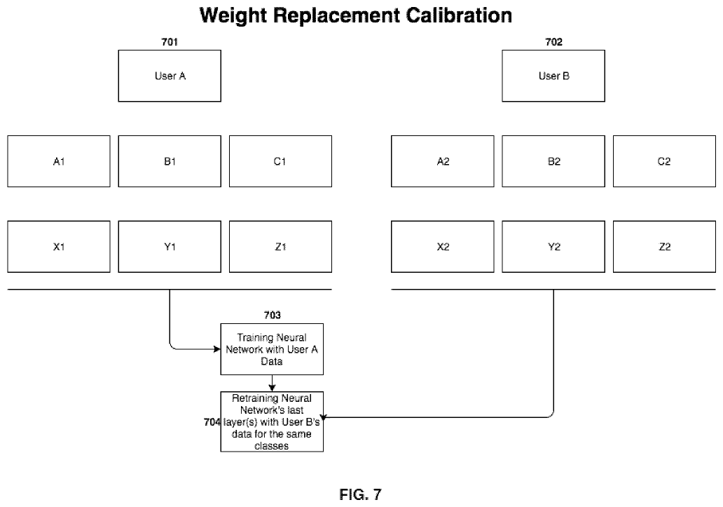

### DETAILED DESCRIPTION

The following will disclose the method of enabling brain-to-text and brain-to-sound in a different manner, not by measuring the areas in the brain that compose speech, but rather by measuring muscle signals that generate speech. That is, by measuring the intent to speak and/or the intent to generate sound before actual muscle movement and before the actual generation of the sound audible by others. The system may also measure the intention to speak and/or to generate sound through minuscule muscle movement, or through full muscle movement purely from taking the muscles signals as an input from the user. The first phase is the training phase, which encompasses methods by which data is collected, source localization, training of deep learning modules and calibration of the system to the user. The second phase comprises sensing brain and/or muscle signals from the user, processing the signals at the deep learning module and converting said signals into an output. The output may be text or automatically generated speech. The user does not need say anything out loud; the user may simply intend to speak; moves their muscles slightly (minuscule muscle movements); or actually move their muscles fully (as if they are speaking out loud). The movements may be voluntary movements such as intended speech, minuscule muscle movements, or fully moving muscles (i.e. as if speaking out loud).

In one instance, the signals being sensed may be muscle signals combined with brain signals that come from the auditory areas of the brain. The brain signals may also come from areas of the brain that compose speech. This system combines muscle signals with signals from the auditory areas of the brain to measure intended speech. For instance, when you say something in your head (speaking to yourself) you also hear yourself saying it. This engages the auditory areas responsible for listening. Thus, when you say something in your head, it is also as if you heard it.

Furthermore, the signals used during data collection in the training phase, only need to come from one user, rather than a plurality of users to provide data during data collection. Thereafter, the calibration step enables the system to adapt to any new user after only being trained by one user which is less arduous, requires less resources and time, and more accurate.

Prior systems to date describe measuring multiple areas that generate muscle signals in order to augment the decoding of the user's actual audible speech (for example measuring muscle signals from the arm to augment the decoder's understanding of the user's intentions from their body language). In the following system, two variant methods can be used to derive signals. The first variant entails signals that are measured only from muscles that are involved in the production of speech, and signals generated from other muscles that are not directly involved in the production of speech are considered noise. The second variant entails signals that are measured only from muscles that are involved in the production of speech, and signals that are measured from the Auditory areas of the brain (rather than the Speech Composition areas of the brain).

Prior systems use traditional techniques in signal processing, that are also known as pre-processing, which are used to prepare the signal prior to passing it to the deep learning module. The present system does not use any of the traditional fixed signal processing algorithms such as ICA/PCA, Butterworth Filtering, it also does not average the signal. Averaging the signal drives the use of classical machine learning algorithms, rather than deep learning algorithms, whereas the latter relies on many samples for training, and averaging all the samples results in one, or only a few samples which is not enough to train deep learning modules properly. Contrary to traditional approaches, the present system also does not use frequency bands (such as alpha, beta, gamma, delta derived through intermediary signal processing steps such as Fourier Transforms), or a percentage of the frequency bands as the main indicator of a user's intention to speak. Similarly, the present system does not require intermediary analysis of variance (ANOVA), multi-variate analysis of variance (MANOVA), or wavelet transforms during intermediary signal processing. That is, the present system sends signals directly to the deep learning module(s), does not use classical machine learning, or use the traditional signal processing techniques. As such, the use of “machine learning” in the presently described system precludes the use of ‘classical’ machine learning algorithms, such as support vector machine, logistic regression, naive bayes. That is, references herein to use of machine learning by the system is referring to deep models.

It may be noted that references herein to traditionally implemented intermediary signal processing steps refers to fixed methods a priori that transform the signal prior to sending it to the machine learning algorithm (i.e. deep learning). Fixed methods such as ANOVA, MANOVA, signal averaging to find evoked response or event-related potentials (ERP). For example, the present system would not need to isolate frequency bands prior to sending the data to the deep learning process. However, the deep learning algorithm itself may find a shared pattern that resembles that, but it finds that pattern more effectively when the method of doing that is not fixed a priori, like using a fast Fourier transform.

Additionally, prior systems to-date require the user to calibrate every time upon use, or frequently. Whereas the present system only requires the user to calibrate one-time during the setup, and after that becomes calibration-less. Meaning after the setup-calibration, it does not have to be frequently calibrated again for the user to perform the same function it was calibrated to do.

Moreover, the present system not only becomes calibration-less, but provides a novel method for calibration.

Moreover, the present system provides another variant of novel calibration method that enables the user to start using the product out of the box, without any calibration.

In terms of calibration, using the approach of averaging signals from plurality of users forces the prior approaches to use a generic algorithm generalized for all users. This so-called “calibration” should not be considered a calibration because it forces the user to go through an arduous process of tailoring it specifically for them. In contrast, the present system provides a novel approach for calibrating. With the present system, every users model is individualized, with little to no setup. The present system has been found to be less computationally intensive, less arduous, commercially scalable, and importantly, more accurate.

Moreover, specific types of neural networks are used to model distribution of data—such as the ADCCNN (Autoregressive Dilated Convolutional Neural Network) and Generative Models, and Transformers.

Source Localization is done here during the training phase to localize the source to specific muscles from which to derive the signals from. This highly improves the integrity of the data that is used to train the deep learning models, which in turn results in much more accurate models that are highly attuned to each individual user they are calibrated for.

Reference to the term signals throughout this specification refers in specific to brain signals, muscle signals and audio signals.

Reference to muscle signals throughout the description refers to signals measured through EMG Sensors (Electromyography), SMG Sensors (Sonomyography), MMG Sensors (mechanomyography) or any other type of sensor used to measure muscle signals. These signals are only measured from the muscles identified above that are included in the production of speech.

Reference to brain signals throughout the description refers to signals measured through EEG Sensors (Electroencephalography), Neural Lace, ECoG, fMRI, MEG, fNIRS, Graphene-based or any other type of sensor user to measure brain signals whether invasively or non-invasively. These signals are only measured from the auditory brain region identified above, and not from the brain region that composes speech.

### Training Phase:

By way of example, during the training phase data needs to be collected from a user in order to train the system on capturing the intention to speak, on capturing minuscule muscle movements and converting them to text and/or sound, and on capturing full muscle movements and converting them to text and/or sound without taking any audio as input during deployment.

In the training phase, the system takes in signals and accompanying labels, which label each and every segment of the signal with the sounds and/or words. The labels can be words, they can be sounds, they can be both sounds and words or a completely different way of labelling the signal. During the deployment phase, when the product is being used by a user, the user may provide the muscle signals as input, or may provide the muscle signals and brain signals as input, and the system will automatically convert the signals into labels similar to the ones provided during training. The system can generalize to producing words and/or sounds that it may not have necessarily been trained on detecting in the training phase. For example, if during training phase the system was trained on signals and text that labelled every segment of the signal, then during deployment the system will take in the user's signal, and provide as an output, text. If during training the signals were labelled with sound, then during deployment the system will take in signals and provide, as an output, sound.

Therefore, in the Training Phase, the first step is setup a Data Collection whereby the Training Module is provided with Signals and Labels as input. Data is then collected while the user is speaking. For example, the user reads sentences out loud while the muscle sensors are positioned firmly on their skin, or slightly touching the skin, or even without touching the skin at all (depending on the sensor used) as long as the sensors can measure the signals. For example, the user reads “She sells seashells by the seashore”.

The training module receives data that comes in the form of Signals and each segment (also known as Epoch) of those signals overlay/are labelled with the sound. During deployment, the system can use input signal and will generate the label (sound).

Similarly, muscle signals may be converted to text. The user reads words out loud with sensors positioned to measure muscle signals. The training module transcribes the words said out loud into text and labels the muscle signals with text, exactly when the signals resembled it.

Similarly, when converting both muscle and brain signals to text and/or sound, the same process is implemented; signals are labelled and provided to the training module, or labelled by the training module itself when data is being collected, or synchronized in the training module after the data has been collected. In one instance, the sounds of words (or text) may be provided to the training module along with its corresponding signal. In another instance, the sounds of words (or text) may be labelled by the training module itself. In yet another instance, the sounds of words (or text) may be labelled prior to being provided to the training module.

### Source Localization:

In order to collect the most accurate and cleanest data for training the machine learning algorithm that is collected during the training session, source localization can (and is preferable to) be implemented. Not localizing the source of the signals derived from the sensors would not completely fail this approach, nevertheless, it is recommended to derive signals specifically from the muscles involved in the production of speech (and from the auditory areas of the brain if brain signals are also being combined with the muscle signals) to achieve maximum efficiency and accuracy. While traditionally, attempts to measure sounds heard in the brain used all many sensor locations of the 10-20 system (most of which are unrelated to auditory areas) or by using all sensors available in the 10-20 system, data coming from brain regions that are not auditory related (and source localized) are considered noise in the present implementation, as provide features that are irrelevant to the end-result, which renders it less accurate. This highly improves the accuracy of the system.

In order to do source localization, the user whose data is being collected during the training session (by way of example, called User A), undergoes an fMRI scan before the training session starts. A 3D Digitization solution such as the Polhemus-Fastrak as an example, is used in order to digitize points on the user's head. The digitized sensor points are co-registered with the brain anatomy of User A using both their fMRI scan and the output of the digitization solution. Inverse Modelling is employed here and one of a variety of techniques such as LORETA, sLORETA, VARETA, LAURA, Shrinking LORETA FOCUSS (SLF), Backus-Gilbert, ST-MAP, S-MAP, SSLOFO, ALF, as well as beamforming techniques, BESA, subspace techniques like MUSIC and methods derived from it, FINES, simulated annealing and computational intelligence algorithms known to persons skilled in the art of signal processing. A major factor for determining which of the techniques to employ depends on whether there is a fixed number of sensors or not. In this case, the source has to be localized to the auditory system of the brain. Similarly for muscle signals, the source is localized to specific muscles by using a 3D digitization solution and then applying inverse modeling or one of the techniques mentioned above is employed to localize the source of the signal to specific muscles. Inverse Modelling techniques mentioned in this paragraph are not exhaustive. Another method of inverse modelling could be used that is not necessarily mentioned by name above.

Once source localization is complete the data is provided to the machine learning module to train the algorithms.

Two embodiments of machine learning techniques will be described below.

In a first embodiment, the machine learning algorithm can classify specific windows of signals as a specific word, or sound which can be done online(meaning in real-time) or offline (meaning after the signal has been given to the system). In the second embodiment, the machine learning algorithm can reconstruct windows of signals into sentences or sounds which can be done online(meaning in real-time) or offline (meaning after the signal has been given to the system).

Although traditional machine learning approaches can be used, Convolutional Neural Networks (CNNs), Generative Transformers, Generative Pre-trained Transformers and Generative Models in general are particularly advantageous for the detection of words and/or sounds from muscle signals, or from muscle and brain signals together, and have achieved an accuracy of over 98% in practice. It can be appreciated that many words can be added by training the deep learning algorithm with more examples of data for different classes (of words/sounds), with the neural network's hyper-parameters and weights optimized accordingly. With more training data, it becomes even more accurate.

Traditionally, EEG and EMG signals are filtered using known signal processing techniques like butter-worth filtering and other techniques such as ICA (Independent Component Analysis), PCA (Principal Component Analysis) which are examples of these techniques. However, the presently described implementation does not employ any of these techniques, while being more effective through this implementation to construct and enable the deep learning algorithm to detect the desired signals rather than resorting to these traditional approaches. Traditional approaches include averaging the signals of each class to find what's known as the Evoked Response (the average signal for a specific class of body movement) or to find Event Related Potentials (ERR), like isolating frequency bands during intermediary signal processing, applying wavelet transformations, and then training an algorithm such as Logistic Regression or other ‘classical machine learning algorithms’.

The present system does not average signals (which reduces the amount of data for training the algorithm, hence requiring data from a plurality of users), as a CNN (as well as other deep learning modules) requires large amount of data from training, but rather optimizes the network to find a shared pattern among all raw training examples provided directly to the network.

There are four variants for training the deep learning module. The first variant is training the CNN or Generative model variant directly from the raw data.

The second variant is constructing an algorithm that first learns the feature representation of the signals through two (or more) different models within the same algorithm rather than just one model. For example, the first stage is a model that learns the features of EEG data, such as a Long-Short-Term-Memory Network (LSTM), which outputs feature vectors for every labelled epoch of data, and provides that output as an input into the second model. The second model is a CNN or Generative Model that receives the feature vectors from the LSTM or Dilated CNN as input and provides the measured classes(of words/text/sound/speech) as output. A CNN can be employed with the first model being a Dilated CNN that learns the features of EEG data over long range temporal dynamics.

The third variant is constructing an Autoregressive Dilated Causal Convolutional Neural Network (ADCCNN) that directly receives signals and adding an optional “student” module to that will allow it to be faster by more than a thousand times when deployed into production. This will be explained in greater detail in the sections below.

The ADCCNN is trained on providing an output of classes that indicates what words or sounds were and silently communicated made by the user (which can happen simultaneously), and indicates that in a sequential manner. Meaning the ADCCNN for the purposes of this capability takes in a sequence of signals and provides as an output a sequence of samples corresponding to what classes or sequences of results such as text, sound, or other media that were detected as being performed by the user.

The fourth variant consist of two tiers that can be used. In the first tier there can be two or more models where the first model is a network that learns and outputs vectors that represent features of EMG data (or EMG and EEG data) of the raw training data provided. As such, learns the features of data for training User A when they are intending to speak, or making minuscule muscle movements to speak, or making full muscle movements to speak. A recurrent neural network, in this case an LSTM or Transformer is found to be ideal, nevertheless that is not a limitation to what type of network can be deployed here to learn features. A second model is constructed that receives the output of the first model and generates text, sound (and can be images or a video) using those features, that is as close as possible (and after extensive training becomes exact) to the original training sounds and or word labels provided to the training module (in the first variant), and when deployed it can reconstruct (regenerate) words and or sounds and data that were not seen during training, when the neural network is trained through the second training variant. This, in essence, teaches this variant to reconstruct (convert) signals into words and sounds (and also images and videos if these were provided as labels to the training module at the start of the training phase). Training through this method overcomes the traditionally known “open problem of words”, which states that there is an unlimited amount of words in the world (as they keep increasing) and that it would be difficult to categorize them all. This overcomes the problem by enabling the network to generate any word or sound without having been specifically trained on recognizing that target label in the training phase. The problem is also overcome in terms of categorizing words, and not only reconstructing them, through the feedback loop between blocks.

The second model of the fourth variant can be a variational Auto-Encoder (VAE), Convolutional Auto-Encoders, or a Generative Adversarial Network (GAN), Deconvolutional Generative Adversarial Networks, Autoregressive Models, Stacked GAN, GAWNN, GAN-INT-CLAS, Generative Pre-trained Transformer, or a variant of any of the above to generate an output from the input features of the first model. Where in this case (a GAN), the feature output of the first model of the network (LSTM) is used as input to the two sides of a GAN—the discriminator and the generator. Where the generator generates target labels (words/sounds/text/image/video) and the discriminator assesses how accurate the generated labels are relative to what it should be from the input label, and provides a feedback loop for the generative portion of the network to improve while the network is being trained.

In the second tier of the fourth variant the process of implementing label generation from muscle signals, or from muscle and brain signals can be implemented as follows:

First, the system constructs a unique model in the field of Silent Communication. The model is based on an ADCCNN applied, which exhibit very large receptive fields to deal with the long ranged temporal dynamics of input data needed to model the distribution of, and generate labels (words/text/image/sounds/videos) from the muscle signals (or from the muscle and brain-signals).

Each sample within an epoch/period of signal data is conditioned by the samples of all previous timestamps in that epoch and epochs before it. The convolutions of the model are causal, meaning the model only takes information from previous data, and does not take into account future data in a given sequence, preserving the order of modelling the data. The predictions provided by the network are sequential, meaning after each sequence is predicted, it is fed back into the network to predict the next sample after that.

Optionally an ‘student’ feed-forward model can be added, rendering a trained ADCCNN to be the teaching model. This is similar to the Generative Adversarial Network, save for the difference being that the student network does not try to fool the teaching network like the generator does with the discriminator. Rather, the student network models the distribution of the ADCCNN, without necessarily producing one sample at a time, which enables the student to produce generations of target labels while operating under parallel processing, producing an output generation in real-time. This enables the present system to utilize both the learning strength of the ADCCNN, and the sampling of the student network, which is advised to be Inverse Autoregressive Flow (IAF). This distills probability distribution learned by the teaching network to the student network, that when deployed into production, can be thousands of times faster than the teaching network at producing the output. This means the result (when adding the student network) can generate from the first to last target label altogether without generating one sample at a time in between, improving output resolution with number of target labels.

Whether tier 1 (a variation of RNN and GAN) is used, or tier 2 (a novel variation of CNNs with an additional student network learning the distribution in a manner that speeds up processing by enabling it to be computed in parallel), the output of either tier I or tier II is the produced target label.

After having trained the algorithm on detecting specific intended words/sounds/labels or converting/reconstructing/generating words/sounds/target-labels (including previously unseen target labels), the system has a pre-trained model that, along with its optimized weights through training, is deployed into the API (application programming interface) for the purposes of decoding silent speech through muscle signals or through muscle signals and brain signals, providing an output to power any application. The API hosts the training module and the deep learning module after deployment.

When a new user starts using this API there is a calibration that is done effectively and in a very short amount of time for any new user of the system.

### Calibration:

The deployed pre-trained deep learning model has learned the features of the EMG (or EMG and EEG) data provided to the training module. More specifically, every layer of the network as the system goes ‘deeper’, meaning to the next layer of the neural network, learns features of the signal that are less abstract and more specific to the muscle (or muscle and brain signals) of the training dataset's user. By way of example, the training dataset here was collected from User A, and User B is a person who will use this technology for the first time. And also by way of example, the network is trained on detecting five silently spoken words by User A.

Then, User B wearing the wearable device and is asked through an interface connected to the API, to silently communicate again the five silently spoken words. This overcomes the problem of different users because there is a vast difference between the training process of User A, and the calibration process of User B. First being that training of the neural network for the first time by User A is very extensive and time consuming, and should be done in a controlled environment such as a lab. User B's calibration is done in a short amount of time (e.g. 15 seconds in the case of five classes), depending on the number of classes s/he is asked to perform.

Calibration can be done in a commercial setting where the user can be anywhere, rather than a controlled environment. It is also significantly less computationally intensive. While training a deep learning network takes days on a normal CPU (Central Processing Unit), or can be [comparatively] trained with a few hours, minutes, or seconds using GPU (Graphical Processing Unit) depending on how many GPU's are utilized for training, it still requires a very intensive computational power to bring the training time down to seconds or less. This approach's requisites are that User B calibrates with a much smaller dataset than was used during training of User A. For example, five samples for each class was found to be more than enough for the mentioned deep learning module to calibrate for User B, while achieving near-perfect accuracy.

The calibration process is done by using the same pre-trained deep learning model with the weights optimized to data derived from User A, but removing the last (final layer) of the network, and replacing it with a new layer re-optimized with weights to User B's signals—see Through this Transfer of Knowledge approach, User B can start using the technology with only a few examples of training, in a very short amount of time, in a commercial setting, and in a computationally efficient manner.

It may be noted that the deeper the network is (greater the number of layers), the more likely that the system would need to re-optimize the last ‘N’ number of layers or more because as mentioned above the more layers go deep, the more they become specific to the data of User A used for initial training. In the CNN mentioned above, removing only the last layer was more efficient than removing the last two.

It may also be noted that due to signal variability between users, User B's brain (if brain signals are being combined with muscle signals) is expected to change over time. Hence, ideally the calibration is advised to be done weekly or bi-weekly (if brain signals are being used) in a very short amount of time to ensure that maximum accuracy is continually achieved. If only muscle signals are being used, calibration has to be done only once, during setup. There is no ideal rate for how often calibration should be done if brain signals are being combined with muscle signals as the neuroplasticity rate is different for each user depending on age and a number of other factors.

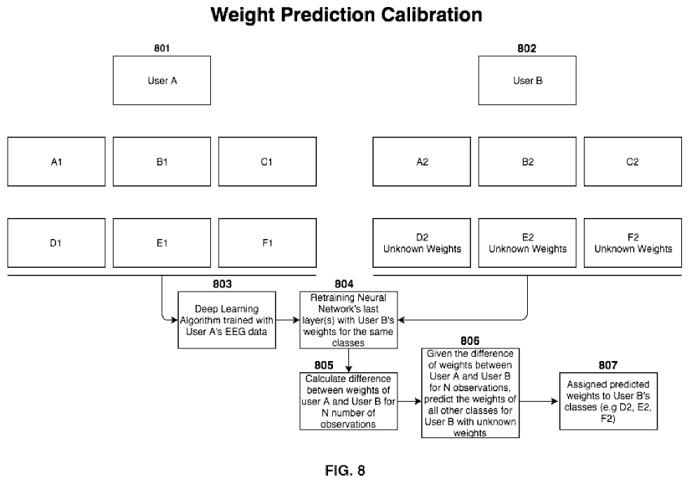

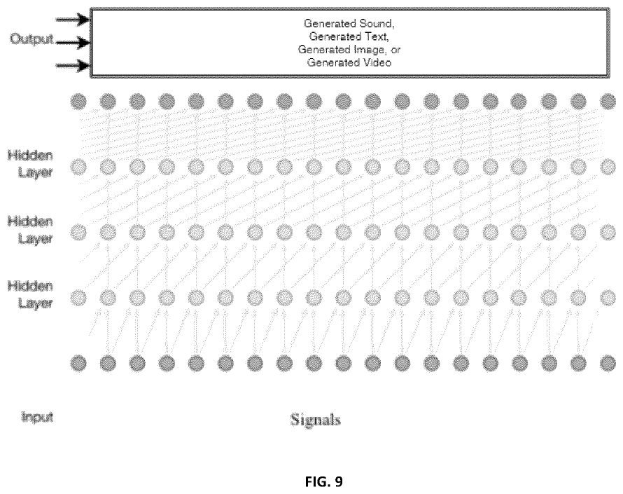

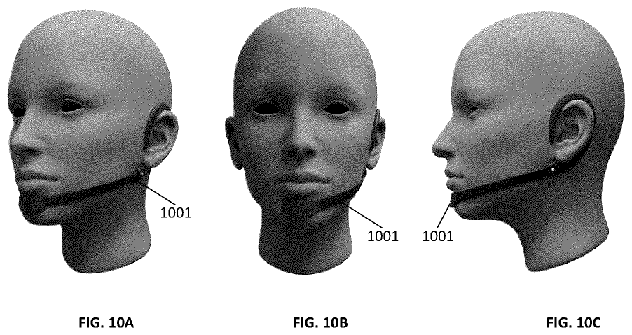

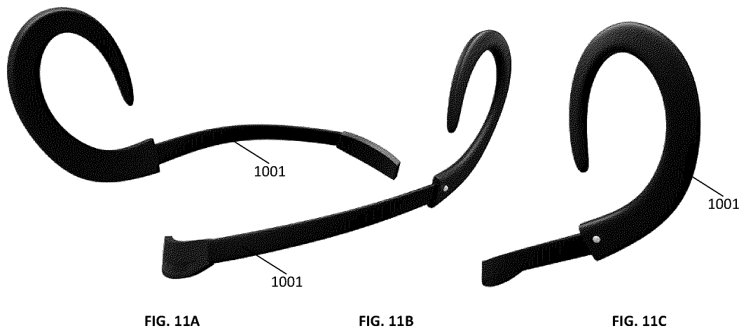

While traditionally any attempt to model a user's silently communicated sounds/words from their muscle signals (or muscle and brain signals) to power an application, has been positioned in a way that when a new user starts using it, it starts learning specifically to their muscles (and brain) from scratch or from a generic baseline, the description here describes two novel calibration methods in FIGS. 8 and 9, and described above which provide many advantages such as calibrating in a short amount of time, being minimally intensive in terms of computation, enables calibration in a commercial setting by any user, and the algorithm does not start learning from scratch, meaning it requires much fewer training examples to calibrate, while maintaining a very high level of accuracy.

Once the API is calibrated to the new User, it will detect a user's silent communication with maximum accuracy which can be used to power many applications.

### Weight Prediction Calibration:

User A provides training data (signals) that are synchronized with labels to the training of the deep learning module.

A machine learning (e.g., deep learning algorithm) is constructed and is trained for classification of the signal windows into words and or sounds. It has been found that RNNs are ideal networks to learn the features of time-series EEG data. This approach is not limited to an LSTM, however, by way of example, an RNN. An LSTM has been found in practice to achieve 90%+accuracy, and can be improved further by adding more data, more layers and optimizing the hyper-parameters of the network and its weights accordingly.

The deep learning model, once trained on EMG (or EMG and EEG) features of raw data can accurately classify a previously unseen word/sound by the user belonging to that same category.

The deep learning model is then deployed within the API along with its weights, ready to receive data and provide as an output what words/sounds the user was intending to say as is detected from their muscle signals (or brain and muscle signals).

A calibration as described above is typically required to calibrate the system from being trained with training data collected by a training user, to a new user in a new setting (commercial setting for example) as seen in FIG. 9. By way of example, User A at block 801 is the training User, and User B at block 802 is a new user of the technology. User B is presented with words on the screen to silently communicate, and the difference in weights between User A's exhibiting of signals when silently communicating the word ‘A’ and User B's exhibiting of signals to that same word ‘A’ is calculated, and this process is done for a number of images. The difference in weights for every image is used to retrain the deep learning model's last layer (or more depending on depth of the model) through the transfer of knowledge method described above.

For example—if the model is trained on recognizing one hundred words silently spoken by User A. When User B starts using the API, they are presented with, by way of example, images of five words. Objects A1, A2, A3, A4, and A5.

The weight of each class as was trained by User A is X1 for A1, X2 for A2, X3 for A3, X4 for A4, and X5 for Word A5.

When User B is presented with and silently communicates the words of the same five words A1, A2, A3, A4, and A5, the last layer (or more) of the network are retrained for User B. The weights for User B are Y1 for A1, Y2 for A2, Y3 for A3, Y4 for A4, and Y5 for A5. Then Weight Prediction is posed to be: Calculate the difference between Y1 and X1 for word A1, Y2 and X2 for word A2, Y3 and X3 for word A3, Y4 and X4 for word A4, and Y5 and X5 for word A5 (see block 805).

Given the difference between X and Y for every word A, predict the weights for all other classes Y6 to Y100 for words A6 to A100, given the known values of X6 to X100, weight prediction calibration is implemented (see block 806).

This calibration approach can enable the deep learning model to adapt to a new user's brain effectively, in a short amount of calibration time, with minimal computational intensity, being viable to be used by a new user in a commercial setting (see block 807).

Many applications of this technology are available. For instance, the system and method may be used for, for instance, enabling stroke patients that lose the ability to speak, to communicate; enabling persons with mute disability to be able to speak; talking to virtual assistants by silent communication; processing credit card payments.

The following will describe applications for this system and device.

Law Enforcement & Private Security: Police Officers are able to silently communicate with one another without being heard by others. This works when the system generates speech or text from the input signals in the deployment phase when being used by the users. Examples of usage in this industry are when entering a building, officers need not communicate with hand gestures, but rather can communicate silently using the technology. Officers can call for backup silently without being detected if they have both their hands in the air at gunpoint. For example, bone conduction can be used by the other users to enable them to listen without impeding their hearing from surroundings.

Gaming: The present system can be used as input into gaming consoles, PCs, and video games of all types. It can be used to give commands to the game as a replacement to keyboard and mouse, it can also be used to augment keyboard and mouse enabling the user to click with their intentions, type with their intentions and also give specific commands to the game.

Underwater Communication: The present system if encapsulated in water-proof enclosure can enable divers and swimmers to communicate underwater.

Replacement to Voice Recognition and Voice-powered applications: Users can use the technology to type with their intentions as a replacement to speaking out loud when communicating with Siri, Alexa, or any other voice-operated technologies that rely on receiving sound from the user.

Enabling mute persons to be able to speak: Persons with disabilities such as being mute or having a speech disorder (aphasia) can use the present system to speak with their intentions or to type with their intentions which enables them to communicate with others and use technologies that otherwise would rely on voice as input.

Persons with disabilities and stroke patients that lose the ability to use their hands or certain muscles or able to use this technology to type with their intentions, control electronic devices and technologies that otherwise would rely on them typing or receiving voice as input.

For simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures, and components have not been described in detail so as not to obscure the examples described herein. Also, the description is not considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

Although the above principles have been described with reference to certain specific examples, various modifications thereof will be apparent to those skilled in the art as outlined in the appended claims.

1. A method of generating speech from human intent comprising:
A training phase comprising:
Collecting data from a human;
Localizing a source;
training deep learning modules; and
A deployment phase comprising:
sensing signals;
processing the signals at the deep learning module; and
converting said signals into an output;
wherein the signals comprise voluntary intentions.

2. The method of claim 1; wherein the voluntary intentions are brain signals.

3. The method of claim 2; wherein the voluntary intentions are muscle signals.

4. The method of claim 3; wherein the voluntary intentions are a combination of brain and muscle signals.

5. The method of claim 4; wherein the output is text or automatically generated speech.

6. The method according to claim 5; wherein the source is localized in auditory areas of the brain.

7. The method according to claim 6; wherein sounds of words are provided to the deep learning modules at the training phase.

8. The method according to claim 7; wherein text corresponding to words is provided to the deep learning modules at the training phase.

9. The method according to claim 8; wherein sounds of words are labelled by the deep learning modules at the training phase.

10. The method according to claim 9; wherein sounds of words are labelled prior to being provided to the deep learning modules at the training phase.

11. A device for generating speech from human intent comprising:
at least one sensor for measuring signals;
a processor;
at least one deep learning module; and
a wearable portion;
wherein the deep learning module is used for training the device and processing the measured signals;
wherein a training phase is used to localize a source to specify muscles from which to derive the signals from;
wherein the processor converts said signals into an output; and
wherein the signals comprise voluntary intentions.

12. The method of claim 11; wherein the voluntary intentions are brain signals.

13. The method of claim 12; wherein the voluntary intentions are muscle signals.

14. The method of claim 13; wherein the voluntary intentions are a combination of brain and muscle signals.

15. The method of claim 14, wherein the output may be text or automatically generated speech.

16. The method according to claim 15; wherein the source is localized in auditory areas of the brain.

17. The method according to claim 16; wherein sounds of words are provided to the deep learning modules at the training phase.

18. The method according to claim 17; wherein text corresponding to words is provided to the deep learning modules at the training phase.

19. The method according to claim 18; wherein sounds of words are labelled by the deep learning modules at the training phase.

20. The method according to claim 19; wherein sounds of words are labelled prior to being provided to the deep learning modules at the training phase.
